(19) United States
(12) Patent Application Publication
(10) Pub. No.: US 2007/0180630 A1
(43) Pub. Date: Aug. 9, 2007

(54) Hair colorants with vat dyes

(75) Inventors: Manuela Javet, Marly (CH); Catherine Mueller, Marly (CH); Anita Roulin, Villarlod (CH)

Correspondence Address: Striker Striker & Stenby, 103 East Neck Road, Huntington, NY, 11743, US

(73) Assignee: WELLA AG, DARMSTADT (DE)

(21) Appl. No.: 10/590,258

(22) PCT Filed: Nov. 24, 2004
(86) PCT No.: PCT/EP04/13305

§ 371 Date: Aug. 22, 2006

(30) Foreign Application Priority Data
Mar. 26, 2004 (DE) 10 2004 014 764.7

(51) Int. Cl. A61K 8/00

(52) U.S. Cl. 8/405

(57) ABSTRACT

The agent for dyeing hair has a pH of 4 to 11 and contains a reduced vat dye, which is obtained by reducing a vat dye, such as an anthrquinonoid dye, with a reducing agent compound that forms an enediol in an alkaline medium, preferably acetoin, monohydroxyacetone, and dihydroxyacetone. The method of dyeing hair includes pre-reducing the vat dye with the reducing agent compound at a pH of from 10 to 13 to form the reduced vat dye, providing an agent for dyeing the hair having a pH of from 4 to 11 and containing the reduced vat dye, allowing the agent to act on the hair while protecting the reduced vat dye in the agent from atmospheric oxygen, and then re-oxidizing with atmospheric oxygen or an oxidant.

The present invention has for an object an agent and a method for dyeing hair, particularly human hair, with so-called vat dyes.

Although vat dyes are widely used for dyeing cellulosic fibrous materials, they are very rarely used for animal fibers, for example wool or hair, because these fibers do not withstand the required very high pH of about 13 without undergoing drastic damage. Moreover, in most cases very aggressive reducing agents are used, for example sodium dithionite (hydrosulfite) or sodium hydrogen sulfite. For these reasons, the dyeing of animal fibers with vat dyes has thus far not been adopted in practice.

In the dyeing of textiles, attempts have already been made to replace hydrosulfite vatting agents with other reducing agents, because, among other things, hydrosulfites contaminate wastewater to a high degree. In EP 0 357 548 A1, enediols have been recommended as an appropriate replacement because under alkaline conditions they reduce the dyes practically as well as does dithionite, but are much better tolerated environmentally. These processes, however, cannot be applied to keratin fibers but only to cellulosic ones, be-cause a pH of about 13 is still required for the dyeing. Alternatively, for hair dyeing it has been proposed in DE 44 27 888 A1 to reduce vat dyes by use of laser light. In practice, however, because of the use of laser radiation, this process is applicable only with difficulty.

The purpose was therefore to provide a method whereby hair can be dyed with vat dyes in a simple and gentle manner.

Surprisingly, we have now found that dyeing with vat dyes (for example indanthrene dyes) can be carried out also under physiologically tolerable conditions by suitably adjusting the pH before dyeing. In this manner, uniform hair colorations are obtained while at the same time the hair is treated as sparingly as possible. Moreover, this method allows so-called brightening dyeing to be achieved with partial or complete decolorization of the natural hair pigment melamine during the final oxidation step.

The present invention has for an object an agent for coloring hair characterized in that it contains at least one vat dye reduced by use of a compound which in alkaline medium forms an enediol, said agent having a pH of 4 to 11.

By vat dyes in the sense of the invention are meant anthraquinonoid dyes and optionally sulfur dyes that have not been pre-reduced. Preferred dyes are, for example, Vat Yellow 1 (C.I. 70600), C.I. Vat Yellow 2, C.I. Vat Yellow 3, C.I. Vat Yellow 4 (C.I. 59100), C.I. Vat Yellow 12 (C.I. 65405), C.I. Vat Yellow 13, C.I. Vat Yellow 17 (C.I. 65415), C.I. Vat Yellow 20 (C.I. 68420), C.I. Vat Yellow 26, C.I. Vat Yellow 28 (C.I. 69000), C.I. Vat Yellow 33, C.I. Vat Yellow 46, C.I. Vat Orange 1 (C.I. 59105), C.I. Vat Orange 2 (C.I. 59705), C.I. Vat Orange 3 (C.I. 59300), C.I. Vat Orange 7, C.I. Vat Orange 9 (C.I. 59700), C.I. Vat Orange 11 (C.I. 70805), C.I. Vat Orange 15 (C.I. 69025), C.I. Vat Orange 17 (C.I. 65415), C.I. Vat Orange 19 (C.I. 59305), C.I. Vat Red 10 (C.I. 67000), C.I. Vat Red 13 (C.I. 70320), C.I. Vat Red 14, C.I. Vat Red 15, C.I. Vat Red 18 (C.I. 60705), C.I. Vat Red 23, C.I. Vat Red 28 (C.I. 65710), C.I. Vat Red 32, C.I. Vat Red 35 (C.I. 68000), C.I. Vat Violet 1 (C.I. 60010), C.I. Vat Violet 10, C.I. Vat Violet 15 (C.I. 63355), C.I. Vat Violet 16 (C.I. 65020), C.I. Vat Blue 4 (C.I. 69800), C.I. Vat Blue 6 (C.I. 69825), C.I. Vat Blue 20 (C.I. 59800), C.I. Vat Blue 21 (C.I. 67920), C.I. Vat Blue 25 (C.I. 70500), C.I. Vat Blue 26 (C.I. 60015), C.I. Vat Blue 29, C.I. Vat Blue 30 (C.I. 67110), C.I. Vat Blue 43, C.I. Vat Blue 64 (C.I. 66730), C.I. Vat Blue 66, C.I. Vat Green 1 (C.I. 59825), C.I. Vat Green 3 (C.I. 69500), C.I. Vat Green 9, C.I. Vat Green 11 (C.I. 69850), C.I. Vat Green 12 (C.I. 70700), C.I. Vat Brown 1 (C.I. 70800), C.I. Vat Brown 3 (C.I. 69015), C.I. Vat Brown 45 (C.I. 59500), C.I. Vat Black 16, C.I. Vat Black 25 (C.I. 69525), C.I. Vat Black 27 (C.I. 69005) or C.I. Vat Black 29 (C.I. 65225) and mixtures thereof. Some of these dyes are marketed by, among others, Dy-star (Frank-furt/Main). Other suitable vat dyes can be found in the Color Index (C.I.), 3rd edition, 1971, published by the Society of Dyers and Colorists, sections on vat dyes and sulfur dyes.

The vat dyes are used in a total amount from 0.01 to 10 weight percent and preferably from 0.1 to 2 weight percent.

Suitable reducing agents are those which in an alkaline medium form enediols (enediolates), for example monohydroxyacetone, dihydroxyacetone, acetoin, glutaroin, adipoin, glycol aldehyde, benzoin, 2,3-dihydroxyacrylaldehyde and cyclopentadiolone, among which acetoin, monohydroxyacetone and dihydroxyacetone are particularly preferred.

Suitable alkalinizing agents for the reduction step are sodium hydroxide and potassium hydroxide or the alkali metal salts of aromatic monohydroxy- and polyhydroxy compounds, for example sodium phenoxide and potassium phenoxide, as well as sodium cresolate and potassium cresolate. The pH for the reduction step is in general 10 to 13.

The reducing agents are used either in an equimolar amount or in up to a 50-fold molar excess, based on the vat dye, a 3-fold to 5-fold molar excess being particularly preferred.

Moreover, the reduction can be carried out in the presence of an organic solvent which is used at a concentration from 0.1 to 80 weight percent and preferably from 5 to 50 weight percent. The organic solvent should be able to form a homogeneous phase with water. Suitable are both protic and aprotic organic solvents, for example ethanol, n-propanol, isopropanol, n-butanol, glycols, for example ethylene glycol and propylene glycol, lactones, amides of the lower carboxylic acids, ureas, sulfones and sulfoxides. Mixtures of these solvents can also be used.

To accelerate the reduction and to improve the absorption of the dye by the fiber, it is optionally possible to use cationic compounds and preferably cationic polymers such as, for example, Polyquaternium-2, Polyquaternium-4, Polyquaternium-5, Polyquaternium-6, Polyquaternium-7, Polyquaternium-10, Polyquaternium-11, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-31, Polyquaternium-35, Polyquaternium-36, Polyquaternium-37, Polyquaternium-39, Polyquaternium-44, Polyquaternium46, Polyquaternium-47, Polyquaternium-51, Polyquaternium-55, Polyquaternium-57, Quaternium-80 Hydroxypropylguarhydroxypropyltrimethylammonium chloride, guar-hydroxypropyltrimethylammonium chloride or mixtures thereof. Particularly preferred are Polyquaternium-4 (hydroxycellulose-dimethyldiallylammonium chloride copolymer), Polyquaternium-7 (dimethyidiallylammonium chloride—acrylamide copolymer and Polyquaternium-22 (acrylic acid-diallyldimethylammonium chloride copolymer).

The cationic compounds are used in a total amount from 0.001 to 5 weight percent and particularly from 0.1 to 1 weight percent.

For the dyeing that follows the reduction step, we found it to be advantageous to add ammonia or ammonium ions, for example in the form of aqueous ammonia or ammonium salts. Amines such as, for example, glucamines, aminomethylpropanol, monoethanolamine and triethanolamine, or inorganic bases, for example ammonium carbonate, ammonium hydrogen carbonate or ammonium carbonate [sic] can also be added.

After the reduction, the strongly alkaline pH of the dye composition is adjusted to a physiologically tolerable value of 4 to 11 and preferably 6 to 10 by use of an acid or of a hydrolyzable substance, for example a salt or an ester. The adjustment to the desired pH can be carried out by use of, for example, buffer systems or inorganic or organic acids, for example lactic, citric, tartaric, glycolic, acetic or phosphoric acid or with ammonium sulfate, ammonium chloride or cetyl lactate.

After the reduction step, it is possible to add to the colorant of the invention common oxidation dye precursors. Suitable oxidation dye precursors are, for example, the following developers, couplers and self-coupling compounds:

(i) Developers: (i) 1,4-diaminobenzene (p-phenylenediamine), 1,4-diamino-2-methylbenzene (p-toluylenediamine), 1,4-diamino-2,6-dimethylbenzene, 1,4-diamino-3,5-di-ethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 1,4-diamino-2-(pyridin-3-yl)-benzene, 2,5-diaminobiphenyl, 1,4-diamino-2-methoxymethylbenzene, 1,4-diamino-2-aminomethylbenzene, 1,4-diamino-2-hydroxymethylbenzene, 1,4-diamino-2-(2-hydroxyethoxy)benzene, 2-[(2-acetylamino)ethoxy]-1,4-diaminobenzene, 4-phenylaminoaniline, 4-dimethylaminoaniline, 4-diethylaminoaniline, 4-dipropylaminoaniline, 4-[ethyl(2-hydroxyethyl)amino]aniline, 4-[di(2-hydroxyethyl)-amino]aniline, 4-[di(2-hydroxyethyl)amino-2-methylaniline, 4-[(2-methoxyethyl)amino]aniline, 4-[(3-hydroxypropyl)amino]aniline, 4-[(2,3-dihydroxypropyl)amino]aniline, 1,4-diamino-2-(2-hydroxyethyl)benzene, 1,4-diamino-2-(1-methylethyl)benzene, 1,3-bis[(4-aminophenyl)(2-hydroxyethyl)amino]-2-propanol, 1,4-bis[(4-aminophenyl)amino]butane, 1,8-bis-(2,5-diaminophenoxy)-3,6-dioxaoctane, 4-aminophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)phenol, 4-amino-2-fluorophenol, 4-amino-2-[(2-hydroxyethyl)amino]methylphenol, 4-amino-2-methylphenol, 4-amino-2-(methoxymethyl)phenol, 4-amino-2-(2-hydroxyethyl)phenol, 5-aminosalicylic acid, 2,5-diaminopyridine, 2,4,5,6-tetraaminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 2-aminophenol, 2-amino-6-methylphenol and 2-amino-5-methylphenol, alone or in admixture with one another.

(ii) Couplers: N-(3-dimethylaminophenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-d iamino-1-ethoxy-5-methylbenzene, 2,4-d iamino-1-(2-hydroxyethoxy)-5-methylbenzene, 2,4-di[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1,3-diamino-4-(2,3-dihydroxypropoxy)benzene, 2,4-diamino-(di(2-hydroxyethoxy]benzene, 1-(2-aminoethoxy)-2,4-diaminobenzene, 2-amino-1-(2-hydroxyethoxy)-4-methylaminobenzene, 2,4-diaminophenoxyacetic acid, 3-[di(2-hydroxyethyl)amino]aniline, 4-amino-2-di[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di(2,4-diaminophenoxy)propane, di(2,4-diaminophenoxy)methane, 1,3-diamino-2,4-dimethoxybenzene, 2,6-bis(2-hydroxyethyl)aminotoluene, 4-hydroxyindole, 3-dimethylaminophenol, 3-diethylaminophenol, 5-amino-2-methylphenol, 5-amino4-fluoro-2-methylphenol, 5-amino4-methoxy-2-methylphenol, 5-amino4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 2-[(3-hydroxyphenyl)amino]acetamide, 5[(2-hydroxyethyl)amino]-4-methoxy-2-methylphenol, 5-[(2-hydroxyethyl)amino]-2-methylphenol, 3[(2-hydroxyethyl)amino]phenol, 3-[(2-methoxyethyl)amino]phenol, 5-amino-2-ethylphenol, 5-amino-2-methoxyphenol, 2-(4-amino-2-hydroxyphenoxy)ethanol, 5-[(3-hydroxypropyl)-amino]-2-methylphenol, 3-[(2,3-dihydroxypropyl)amino]-2-methylphenol, 3-[(2-hydroxyethyl)amino]-2-methylphenol, 2-amino-3-hydroxypyridine, 5-amino-4-chloro-2-methylphenol, 1-naphthol, 2-methyl-1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, 1,3-dihydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 3,4-methylenedioxyphenol, 3,4-methylenedioxyaniline, 5-[(2-hydroxyethyl)amino]-1,3-benzodioxol, 6-bromo-1-hydroxy-3,4-methylenedioxybenzene, 3,4-di-aminobenzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-dihydroxyindole, 5,6-dihydro-xyindoline, 5-hydroxyindole, 6-hydroxyindole, 7-hydroxyindole and 2,3-indolinedione, alone or in admixture with one another.

(iii) Self-coupling compounds: 2-amino-5-methylphenol, 2-amino-6-methylphenol, 2-amino-5-ethoxyphenol and 2-propylamino-5-aminopyridine.

The agent of the invention contains the oxidation dye precursors in a total amount from about 0.01 to 12 weight percent and particularly from about 0.2 to 6 weight percent.

For the purpose of attaining certain color shades, the colorant can also contain common natural and/or synthetic direct dyes, for example the so-called vegetable dyes such as, for example, henna, triphenylmethane dyes, aromatic nitro dyes, azo dyes, quinone dyes and cationic or anionic dyes.

Suitable synthetic dyes are, for example 1,4-bis[(2-hydroxyethyl)amino]-2-nitrobenzene, 1-(2-hydroxyethyl)amino-2-nitro-4-[di(2-hydroxyethyl)amino]benzene (HC Blue No. 2), 1-amino-3-methyl-4-[(2-hydroxyethyl)amino]-6-nitrobenzene (HC Violet No. 1), 4-[ethyl-(2-hydroxyethyl)amino]-1-[(2-hydroxyethyl)amino]-2-nitrobenzene hydrochloride (HC Blue No. 12), 4-[di(2-hydroxyethyl)amino]-1-[(2-methoxyethyl)amino-2-nitrobenzene (HC Blue No. 11), 1-](2,3-dihydroxypropyl)amino]-4-[methyl-(2-hydroxyethyl)amino]-2-nitrobenzene (HC Blue No. 10), 1-[(2,3-dihydroxypropyl)amino]-4-[ethyl-(2-hydroxyethyl)amino]-2-nitrobenzene hydrochloride (HC Blue No. 9), 1-(3-hydroxypropylamino)-4-[di(2-hydroxyethyl)amino]-2-nitrobenzene (HC Violet No. 2), 1-methylamino4-[methyl-(2,3-dihydroxypropyl)amino]-2-nitrobenzene (HC Blue No. 6), 2-[(4-amino-2-nitrophenyl)amino]-5-dimethylaminobenzoic acid (HC Blue No. 13), 1-amino-4-[(2-hydroxyethyl)amino]-2-nitroben-zene (HC Red No. 7), 2-amino-4,6-dinitrophenol, 4-amino-2-nitrodiphenylamine (HC Red No. 1), 1-amino-4-[di(2-hydroxyethyl)amino]-2-nitrobenzene hydrochloride (HC Red No. 13), 1-amino-5-chloro-4-[(2-hydroxyethyl)amino]-2-nitrobenzene, 4-amino-1-[(2-hydroxyethyl)amino]-2-nitrobenzene (HC Red No. 3), 4-amino-3-nitrophenol, 4-[(2-hydroxyethyl)-amino]-3-nitrophenol, 1-[(2-aminoethyl)amino]-4-(2-hydroxyethoxy)-2-nitrobenzene (HC Orange No. 2), 4-(2,3-dihydroxypropoxy)-1-[(2-hydroxyethyl)amino]-2-nitrobenzene (HC Orange No. 3), 1-amino-5-chloro-4-[(2,3-dihydroxypropyl)amino]-2-nitrobenzene (HC Red No. 10), 5-chloro-1,4-[di(2,3-dihydroxypropyl)amino]-2-nitrobenzene (HC Red No. 11), 2-[(2-hydroxyethyl)amino]-4,6-dinitrophenol, 4-ethylamino-3-nitrobenzoic acid, 2-[(4-amino-2-nitrophenyl)amino]benzoic acid, 2-chloro-6-ethylamino-4-nitrophenol, 2-amino-6-chloro-4-nitrophenol, 4-[(3-hydroxypropyl)amino]-3-nitrophenol, 2,5-diamino-6-nitropyridine, 1,2,3,4-tetrahydro-6-nitroquinoxaline, 7-amino-3,4-dihydro-6-nitro-2H-1,4-benzoxazine (HC Red No. 14), 1-amino-2-[(2-hydroxyethyl)amino]-5-nitrobenzene (HC Yellow No. 5), 1-(2-hydroxyethoxy)-2[(2-hydroxyethyl)amino]-5-nitrobenzene (HC Yellow No. 4), 1-[(2-hydroxyethyl)amino]-2-nitrobenzene (HC Yellow No. 2), 2-[(2-hydroxyethyl)amino]-1-methoxy-5-nitrobenzene, 2-amino-3-nitrophenol, 1-(2-hydroxyethoxy)-3-methylamino4-nitrobenzene, 2,3-(dihydroxypropoxy)-3-methylamino-4-nitrobenzene, 2-[(2-hydroxyethyl)amino]-5-nitrophenol (HC Yellow No. 11), 3-[(2-aminoethyl)amino]-1-methoxy-4-nitrobenzene hydrochloride (HC Yellow No. 9), 1-[(2-ureidoethyl)amino]-4-nitrobenzene. 4-[(2,3-dihydroxypropyl)amino]-3-nitro-1-trifluoromethylbenzene (HC Yellow No. 6), 1-chloro-2,4-bis-[(2)amino]-5-nitrobenzene (HC Yellow No. 10), 4-[(2-hydroxyethyl)amino]-3-nitro-1-methylbenzene, 1-chloro-4-[(2-hydroxyethyl)amino]-3-nitrobenzene (HC Yellow No. 12), 4-[(2-hydroxyethyl)amino]-3-nitro-1-trifluoromethylbenzene (HC Yellow No. 13), 4-[(2-hydroxyethyl)amino]-3-nitrobenzonitrile (HC Yellow No. 14), 4-[(2-hydroxyethyl)-amino]-3-nitrobenzamide (HC Yellow No. 15), 1,4-di-[(2,3-dihydroxypropyl)amino]-9,10-anthraquinone, 1-[(2-hydroxyethyl)amino]-4-methylamino-9,10-anthraquinone (C.I. 61505, Disperse Blue No. 3), 2-[(2-aminoethyl)amino]-9,10-anthraquinone (HC Orange No. 5), (1-hydroxy-4-[(4-methyl-2-sulfophenyl)amino]-9,10-anthraquinone, 1-[(3-aminopropyl)amino]-4-methylamino-9,10-anthraquinone (HC Blue No. 8), 1-[(3-aminopropyl)amino]-9,10-anthraquinone (HC Red No. 8), 1,4-diamino-2-methoxy-9,10-anthraquinone (C.I. 62015, Disperse Red No. 11, Solvent Violet No. 26), 1,4-dihydroxy-5,8-bis[(2-hydroxyethyl)-amino]-9,10-anthraquinone (C.I. 62500, Disperse Blue No. 7, Solvent Blue No. 69), 9-(dimethylamino)benzo[a]phenoxazin-7-ium chloride (C.I. 51175, Basic Blue No. 6), di[4-(diethylamino)phenyl][4-(ethylamino)naphthyl]carbenium chloride (C.I. 42595; Basic Blue No. 7), 3,7-di(dimethylamino)phenothiazin-5-ium chloride (C.I. 52015; Basic Blue No. 9), di[4-(dimethylamino)phenyl][4-(phenylamino)naphthyl]carbenium chloride (C.I. 44045; Basic Blue No. 26), 2-[(4-(ethyl(2-hydroxyethyl)amino)phenyl)azo]-6-methoxy-3-methyl-benzothiazolium methylsulfate (C.I. 11154; Basic Blue No. 41), 8-amino-2-bromo-5-hydroxy-4-imino-6-{[3-(trimethylammonio)phenyl]amino}-1(4H)-naphthalenone chloride (C.I. 56059; Basic Blue No. 99), bis[4-(dimethylamino)phenyl][4-(methylamino)phenyl]carbenium chloride (C.I. 42535; Basic Violet No. 1), tris[4-(dimethylamino)phenyl]carbenium chloride (C.I. 42555; Basic Violet No. 3), 2-[3,6-(diethylamino)dibenzopyranium-9-yl]benzoyl chloride (C.I. 45170; Basic Violet No. 10), di(4-aminophenyl)(4-amino-3-methylphenyl)carbenium chloride (C.I. 42510; Basic Violet No. 14), 1,3-bis[(2,4-diamino-5-methylphenyl)azo]-3-methylbenzene (C.I. 21010; Basic Brown No. 4), 1-[(4-amino-phenyl)azo]-7-trimethylammonio)-2-naphthol chloride (C.C. 12250; Basic Brown No. 16), 1-[(4-amino-2-nitrophenyl)azo]-7-(trimethylammonio)-2-naphthol chloride (Basic Brown No. 17), 1-[(4-amino-3-nitrophenyl)-azo]-7-(trimethylammonio)-2-naphthol chloride (C.I. 12251; Basic Brown No. 17) [sic], 3,7-diamino-2,8-dimethyl-5-phenylphenazinium chloride (C.I. 50240; Basic Red No. 2), 1,4-dimethyl-5-{[4-(dimethylamino)phenyl]azo)}-1,2,4-triazolium chloride (C.I. 11055; Basic Red No. 22), 2-hydroxy-1-[(2-methoxyphenyl)azo]-7-(trimethylammonio)naphthalene chloride (C.I. 12245; Basic Red No. 76), 2-{2-[(2,4-di-methoxyphenyl)amino]ethenyl}-1,3,3-trimethyl-3H-indol-1-ium chloride (C.I. 48055; Basic Yellow No. 11), 3-methyl-1-phenyl-4[(3-(trimethylammonio)phenyl]azo}pyrazol-5-one chloride (C.I. 12719; Basic Yellow No. 57), bis[4-(diethylamino)phenyl]phenylcarbenium hydrogen sulfate (1:1) (C.I. 42040; Basic Green No. 1), 1-[(2-hydroxyethyl)amino]-3-methyl-4-[(4-nitrophenyl)azo]benzene (C.I. 11210; Disperse Red No. 17), 4-[(4-amino-phenyl)azo]-1-[di(2-hydroxyethyl)amino]-3-methylbenzene (HC Yellow No. 7), 2,6-diamino-3-[(pyridin-3-yl)azo]pyridine, 6-hydroxy-5-[(4-sulfophenyl)azo]-2-naphthalenesulfonic acid disodium salt (C.I. 15985; Food Yellow No. 3; FD&C Yellow No. 6), 2,4-dinitro-1-naphthol-7-sulfonic acid disodium salt (C.I. 10316; Acid Yellow No. 1; Food Yellow No. 1), 2-(indan-1,3-dion-2-yl)quinoline-x,x-sulfonic acid (mixture of mono- and disulfonic acid) (C.I. 47005; D&C Yellow No. 10; Food Yellow No. 13; Acid Yellow No. 3), 5-hydroxy-1-(4-sulfophenyl)-4-[(4-sulfophenyl)azo]-pyrazole-3-carboxylic acid trisodium salt (C.I. 19140; Food Yellow No. 4; Acid Yellow No. 23), 9-(carboxyphenyl-6-hydroxy-3H-xanthen-3-one (C.I. 45350; Acid Yellow No. 73, Acid Yellow No. 8), 5[(2,4-dinitrophenyl)amino]-2-phenylaminobenzenesulfonic acid sodium salt (C.I. 10385; Acid Orange No. 3), 4-[(2,4-dihydroxyphenyl)azo]benzenesulfonic acid monosodium salt (C.I. 14270; Acid Orange No. 6), 4-[(2-hydroxynaphth-1-yl)azo]benzenesulfonic acid sodium salt (C.I. 15510; Acid Orange No. 7), 4-[(2,4-dihydroxy-3-[(2,4-dimethylphenyl)azo]phenylazo]benzenesulfonic acid sodium salt (C.I. 20170; Acid Orange No. 24), 4-hydroxy-3-[(sulfonaphth-1-yl)azo]-1-naphthalenesulfonic acid disodium salt (C.I. 14720; Acid Red No. 14), 6-hydroxy-5-[(4-sulfonaphth-1-yl)azo]-2,4-naphthalenedisulfonic acid trisodium salt (C.I. 16255; Ponceau 4R; Acid Red No. 18), 3-hydroxy-4-[(4-sulfonaphth-1-yl)azo]-2,7-naphthalenedisulfonic acid trisodium salt (C.I. 16185; Acid Red No. 27), 8-amino-1-hydroxy-2-(phenylazo)-3,6-naphthalenedisulfonic acid disodium salt (C.I. 17200; Acid Red No. 33), 5-(acetylamino)4-hydroxy-3-[(2-methylphenyl)azo]-2,7-naphthalenedisulfonic acid disodium salt (C.I. 18065; Acid Red No. 35), 2-(3-hydroxy-2,4,5,7-tetraiododibenzopyran-6-on-9-yl)benzoic acid disodium salt (C.I. 45430; Acid Red No. 51), N-[6-(diethylamino)-9-(2,4-disulfophenyl)-3H-xanthen-3-ylidene]-N-ethylethanammonium hydroxide, inner salt, sodium salt (C.I. 45100; Acid Red No. 52), 8-{[4-(phenylazo)phenyl]azo}7-naphthol-1,3-disulfonic acid disodium salt (C.I. 27290; Acid Red No. 73), 2′,4′,5′,7′-tetrabromo-3′,6′-dihydroxy-spiro{isobenzofuran-1(3H),9′[9H]xanthen}-3-one disodium salt (C.I. 45380; Acid Red No. 87), 2′,4′,5′,7′-tetrabromo-4,5,6,7-tetrachloro-3′,6′-dihydroxyspiro{isobenzofuran-1-(3H), 9′-[9H]-xanthen-3-one disodium salt (C.I. 45410; Acid Red No. 92), 3′,6′-dihydroxy-4′, 5′-di-iodospiro-[isobenzofuran-1(3H),9′(9H)-xanthen]-3-one disodium salt (C.I. 45425; Acid Red No. 95), (2-sulfophenyl)di[4-(ethyl((4-sulfophenyl)methyl)amino)phenyl]carbenium di-sodium salt, betaine (C.I. 42090; Acid Blue No. 9; FD&C Blue No. 1), 1,4-bis[(2-sulfo-4-methylphenyl)amino]-9,10-anthraquinone disodium salt (C.I. 61570; Acid Green No. 25), bis[4-(dimethylamino)phenyl]-(3,7-disulfo-2-hydroxynaphth-1-yl)carbenium inner salt, monosodium salt (C.I. 44090; Food Green No. 4; Acid Green No. 50), bis[4-(diethylamino)-phenyl](2,4-disulfophenyl)carbenium inner salt, sodium salt (2:1) (C.I. 42045; Food Blue No. 3; Acid Blue No. 1), bis[4-(diethylamino)phenyl](5-hydroxy-2,4-disulfophenyl)carbenium inner salt, calcium salt (2:1) (C.I. 42051; Acid Blue No. 3), 1-amino-4-(cyclohexyl-amino)-9,10 anthraquinone-2-sulfonic acid sodium salt (C.I. 62045; Acid Blue No. 62), 2-(1,3-dihydro-3-keto-5-sulfo-2H-indol-2-ylidene)-2,3-dihydro-3-keto-1 H-indol-5-sulfonic a disodium salt (C.I. 73015), Acid Blue No. 74), 9-(2-carboxyphenyl)-3-[(2-methylphenyl)-amino]-6-[(2-methyl-4-sulfophenyl)amino]xanthylium inner salt, monosodium salt (C.I. 45190; Acid Violet No. 9), 1-hydroxy-4-[(4-methyl-2-sulfophenyl)amino]-9,10-anthraquinone sodium salt (C.I. 60730; D&C Violet No. 2; D&C Violet No. 43), bis{3-nitro-4-[(4-phenylamino)-3-sulfophenylamino]phenyl}sulfone (C.I. 10410; Acid Brown No. 13), 5-amino-4-hydroxy-6-[(4-nitrophenyl)azo]-3-(phenylazo)-2,7-naphthalenedisulfonic acid di-sodium salt (C.I. 20470; Acid Black No. 1), 3-hydroxy-4-[(2-hydroxynaphth-1-yl)azo]-7-nitro-1-naphthalenesulfonic acid chromium complex (3:2) (C.I. 15711; Acid Black No. 52), 3-[(2,4-dimethyl-5-sulfophenyl)azo]-4-hydroxy-1-naphthalenesulfonic acid disodium salt (C.I. 14700; Food Red No. 1; FD&C Red No. 4), 4-(acetylamino)-5-hydroxy-6-[(7-sulfo-4-[(4-sulfophenyl)azo]naphth-1-yl)azo]-1,7-naphthalenedisulfonic acid tetrasodium salt (C.I. 28440; Food Black No. 1) and 3-hydroxy4-(3-methyl-5-keto-1-phenyl-4,5-dihydro-1H-pyrazol-4-ylazo)naphthalene-1-sulfonic acid sodium salt, chromium complex (Acid Red No. 195), alone or in combination with one another.

The total amount of direct dyes in the agent of the invention is about 0.01 to 7 weight percent and preferably about 0.2 to 4 weight percent.

Other known and common dyes for hair dyeing that can be contained in the colorant of the invention are described in, among other publications, E. Sagarin, “Cosmetics, Science and Technology”, Interscience Publishers Inc., New York (1957), pages 503 ff, in H. Janistin, “Handbuch der Kosmetika und Riechstoffe” [Handbook of Cosmetics and Fragrances], vol. 3 (1973), pages 388 ff, and in K. Schrader, “Grundlagen und Rezepturen der Kosmetika” [Fundamentals and Formulations of Cosmetics], 2nd edition (1989), pages 782-815.

Moreover, the agent of the invention can contain antioxidants such as, for example, ascorbic acid, thioglycolic acid and sodium sulfite as well as agents for complexing heavy metals, for example an ethylenediamine tetraacetate or nitriloacetic acid, in an amount of up to about 0.5 weight percent. Perfume oils can be contained in the dye carrier composition of the invention in an amount of up to about 1 weight percent. Naturally, the afore-described hair colorant can optionally contain other additives commonly used in hair colorants, for example thickeners, for example homopolymers of acrylic acid, vegetable gums, cellulose derivatives and starch derivatives, algae polysaccharides, amphiphilic associative thickeners, furthermore preservatives, antioxidants, for example sodium sulfite, thioglycolic acid or ascorbic acid; complexing agents; solvents such as water or the afore-described lower aliphatic alcohols with 1 to 4 carbon atoms, such as ethanol, propanol and isopropanol, or glycols such as glycerol and 1,2-propylene glycol, or sorbitol, and wetting agents, or emulsifiers from the classes of anionic, cationic, amphoteric or nonionic surface-active substances and furthermore softeners; vaseline, silicone oils, paraffin oil, polysorbates and fatty acids as well as hair-care agents, such as the cationic polymers or resins, lanolin derivatives, cholesterol, vitamins, pantothenic acid, betaine and salts, such as sodium chloride or sodium sulfate. Said constituents are used in amounts commonly employed for such purposes, for example the wetting agents and emulsifiers at a concentration from 0.1 to 30 weight percent and the hair-care agents at a concentration from 0.1 to 5.0 weight percent.

The agent of the invention is preferably formulated in the form of an aqueous or aqueous-alcoholic preparation, for example as a thickened solution or as an emulsion, cream, aerosol foam or gel.

If possible, the reduced colorant preparation is to be stabilized in the reduced form and protected from atmospheric oxygen. This can be accomplished by the use of appropriate packaging, protective gas and addition of protective and/or stabilizing substances or by esterification (so-called leuko vat dye esters prepared with chlorosulfonic acid). Protective or stabilizing compounds are, for example, cationic compounds, for example polymers, surfactants or metal ions, for example zinc ions.

To achieve a uniformly intense coloration of the hair, it is advantageous to protect the reduced form of the dye from atmospheric oxygen even during the dyeing process. This can be done, for example, by covering the moist hair with a plastic cap having valve openings distributed over its entire surface. In this manner, it is possible to “spray” under the cap through the 5 to 10 valve openings a relatively fluid dye mixture and then thoroughly distribute it and massage it in from the outside. A plastic cap with small openings is also useful when with the aid of a filling nozzle an aerosol foam is introduced under the cover and placed onto the hair, after which the foam is distributed by massaging. More-over, for oxidation-sensitive formulations it is possible to apply a dye mixture and then cover it with, for example, a plastic cap, or expose the hair to slow air oxidation without covering it.

After the colorant has acted for about 1 to 60 minutes and preferably for 5 to 30 minutes at about 15 to 60° C. and preferably at 20 to 45° C., the coloration is developed “oxidatively”. Suitable oxidants for developing the coloration are besides simple air oxidation particularly hydrogen peroxide or a compound of addition thereof to urea, melamine or sodium borate, in the form of a 1% to 12% and preferably 1.5% to 6% aqueous solution. The mixing ratio of dye to oxidant depends on the concentration of the oxidant and usually amounts to about 5:1 to 1:2 and preferably 1:1, the amount of oxidant in the resulting mixture of dye and oxidant preferably being from 0.5 to 8 weight percent and particularly from 1 to 4 weight percent. The oxidant is allowed to act until the dye is once again in its insoluble pigment form (usually for about 2 to 5 minutes) and is thus fixed on the hair in washing-resistant manner. The treatment time can, of course, be extended depending on the degree of brightening desired. If the natural pigment is to be brightened by more than two brightening degrees, it is possible also to use a persulfate or a mixture of a persulfate and hydrogen peroxide or an addition compound thereof. Alkaline persulfate/peroxide preparations are preferably used in this case.

Following the dyeing procedure, the hair is rinsed with water and dried. Optionally, the hair can additionally be washed with a shampoo and possibly post-rinsed with an acidic conditioner. The hair is then dried.

The following examples will explain the subject matter of the invention in greater detail without limiting it to these examples.

### EXAMPLES

### Example 1

### Dye Mixture

| | | |
|---|---|---|
| 10.0 | g | of propylene glycol |
| 1.0 | g | of Yellow 5GF (C.I. Vat Yellow 46) |
| 12.0 | g | of sodium hydroxide (10% aqueous solution) |
| 3.0 | g | of sodium chloride |
| 3.0 | g | of acetoin |
| 68.5 | g | of water |

The afore-said substances were mixed and allowed to stand for 20 min at 40° C. The pH was then adjusted to 9.9 with 2.5 g of lactic acid (90% aqueous solution).

10 g of the mixture obtained in this manner was applied to bleached natural hair. The hair was then covered with a plastic cap or plastic sheet. After a treatment time of 30 min at 40° C., 10 g of a 6% aqueous hydrogen peroxide emulsion was massaged in and allowed to act for 5 minutes. The hair was rinsed with water, then with an acidic conditioner and finally dried.

The hair assumed a uniform bright-yellow color.

### Example 2

### Dye Mixture

| | | |
|---|---|---|
| 10.0 | g | of propylene glycol |
| 1.0 | g | of Golden Yellow RK (C.I. Vat Orange 1; C.I. 71105) |
| 12.0 | g | of sodium hydroxide (10% aqueous solution) |
| 3.0 | g | of sodium chloride |
| 3.0 | g | of acetoin |
| 68.5 | g | of water |

The afore-said substances were mixed and allowed to stand for 20 min at 40° C. The pH was then adjusted to 10.0 with 2.5 g of lactic acid (90% aqueous solution).

10 g of the mixture obtained in this manner was applied to bleached natural hair. The hair was then covered with a plastic cap or plastic sheet. After a treatment time of 30 min at 40° C., 10 g of a 6% aqueous hydrogen peroxide solution was massaged in and allowed to act for 5 minutes. The hair was rinsed with water, then with an acidic conditioner and finally dried.

The hair assumed a uniform golden-yellow color.

### Example 3

### Dye Mixture

| | | |
|---|---|---|
| 10.0 | g | of propylene glycol |
| 1.0 | g | of Brilliant Red LGG (C.I. Vat Red 32) |
| 12.0 | g | of sodium hydroxide (10% aqueous solution) |
| 3.0 | g | of sodium chloride |
| 3.0 | g | of acetoin |
| 68.5 | g | of water |

The afore-said substances were mixed and allowed to stand for 20 min at 40° C. The pH was then adjusted to 10.0 with 2.5 g of lactic acid (90% aqueous solution).

10 g of the mixture obtained in this manner was applied to bleached natural hair. The hair was then covered with a plastic cap or plastic sheet. After a treatment time of 30 min at 40° C., 10 g of a 6% aqueous hydrogen peroxide solution was massaged in and allowed to act for 52 [sic] minutes. The hair was rinsed with water, then with an acidic conditioner and finally dried.

The hair assumed a uniform red color.

### Example 4

### Dye Mixture

| | | |
|---|---|---|
| 10.0 | g | of propylene glycol |
| 1.0 | g | of Brilliant Green FBB (C.I. Vat Green 1; C.I. 59825) |
| 12.0 | g | of sodium hydroxide (10% aqueous solution) |
| 3.0 | g | of sodium chloride |
| 0.1 | g | of ethylenediaminetetraacetic acid disodium salt |
| 3.0 | g | of acetoin |
| 68.4 | g | of water |

The afore-said substances were mixed and allowed to stand for 35 min at 40° C. The pH was then adjusted to 10.0 with 2.5 g of lactic acid (90% aqueous solution).

10 g of the mixture obtained in this manner was applied to bleached natural hair. The hair was then covered with a plastic cap or plastic sheet. After a treatment time of 30 min at 40° C., 10 g of a 6% aqueous hydrogen peroxide solution was massaged in and allowed to act for 5 minutes. The hair was rinsed with water, then with an acidic conditioner and finally dried.

The hair assumed a uniform green color.

### Example 5

### Dye Mixture

| | | |
|---|---|---|
| 10.0 | g | of propylene glycol |
| 1.0 | g | of Blue CLF (C.I. Vat Blue 66) |
| 12.0 | g | of sodium hydroxide (10% aqueous solution) |
| 3.0 | g | of sodium chloride |
| 0.1 | g | of ethylenediaminetetraacetic acid disodium salt |
| 3.0 | g | of acetoin |
| 68.4 | g | of water |

The afore-said substances were mixed and allowed to stand for 35 min at 40° C. The pH was then adjusted to 10.0 with 2.5 g of lactic acid (90% aqueous solution). 10 g of the mixture obtained in this manner was applied to bleached natural hair. The hair was then covered with a plastic cap or plastic sheet. After a treatment time of 30 min at 40° C., 10 g of a 6% aqueous hydrogen peroxide solution was massaged in and allowed to act for 5 minutes. The hair was rinsed with water, then with an acidic conditioner and finally dried.

The hair assumed a uniform blue color.

### Example 6

| | | |
|---|---|---|
| 10.0 | g | of propylene glycol |
| 1.0 | g | of Golden Yellow RK (C.I. Vat Orange 1; C.I. 71105) |
| 12.0 | g | of sodium hydroxide (10% aqueous solution) |
| 3.0 | g | of sodium chloride |
| 0.1 | g | of ethylenediaminetetraacetic acid disodium salt |
| 3.0 | g | of acetoin |
| 58.4 | g | of water |

The afore-said substances were mixed and allowed to stand for 20 min at 40° C. The pH was then adjusted to 9.8 with 2.5 g of ammonium sulfate in 10 g of water.

10 g of the mixture obtained in this manner was applied to bleached natural hair. The hair was then covered with a plastic cap or plastic sheet. After a treatment time of 30 min at 40° C., 10 g of a 6% aqueous hydrogen peroxide solution was massaged in and allowed to act for 2 minutes. The hair was rinsed with water, then with an acidic conditioner and finally dried.

The hair assumed a uniform yellow color.

### Example 7

### Dye Mixture

| | | |
|---|---|---|
| 10.0 | g | of propylene glycol |
| 1.0 | g | of Golden Yellow RK (C.I. Vat Orange 1; C.I. 71105) |
| 12.0 | g | of sodium hydroxide (10% aqueous solution) |
| 3.0 | g | of Polyquaternium-7 (8% aqueous solution) |
| 3.0 | g | of sodium chloride |
| 0.1 | g | of ethylenediaminetetraacetic acid disodium salt |
| 3.0 | g | of acetoin |
| 65.4 | g | of water |

The afore-said substances were mixed and allowed to stand for 20 min at 40° C. The pH was then adjusted to 9.8 with 2.5 g of lactic acid (90% aqueous solution).

10 g of the mixture obtained in this manner was applied to bleached natural hair. The hair was then covered with a plastic cap or plastic sheet. After a treatment time of 30 min at 40° C., 10 g of a 6% aqueous hydrogen peroxide solution was massaged in and allowed to act for 2 minutes. The hair was rinsed with water, then with an acidic conditioner and finally dried.

The hair assumed a uniform yellow color.

### Example 8

### Dye Mixture

| | | |
|---|---|---|
| 58.0 | g | of glycerol (86% aqueous solution) |
| 1.0 | g | of Brilliant Green FBB (C.I. Vat Green 1; C.I. 59825) |
| 23.4 | g | of water |
| 12.0 | g | of sodium hydroxide (10% aqueous solution) |
| 0.1 | g | of ethylenediaminetetraacetic acid disodium salt |
| 3.0 | g | of acetoin |

The afore-said substances were mixed and allowed to stand for 1.5 hours at 40° C. The pH was then adjusted to 9.8 with 2.5 g of 90% lactic acid.

10 g of the mixture obtained in this manner was applied to bleached natural hair. The hair was then covered with a plastic cap or plastic sheet. After a treatment time of 30 min at 40° C., 10 g of a 6% aqueous hydrogen peroxide solution was massaged in and allowed to act for 2 minutes. The hair was rinsed with water, then with an acidic conditioner and finally dried.

The hair assumed a uniform green color.

### Example 9

### Dye Mixture

| | | |
|---|---|---|
| 10.00 | g | of propylene glycol |
| 1.00 | g | of Golden Yellow RK (C.I. Vat Orange 1; C.I. 71105) |
| 12.00 | g | of sodium hydroxide (10% aqueous solution) |
| 0.1 | g | of ethylenediaminetetraacetic acid disodium salt |
| 3.00 | g | of acetoin |
| 71.4 | g | of water |

The afore-said substances were mixed and allowed to stand for 20 min at 40° C. The pH was then adjusted to 10.0 with 2.5 g of lactic acid (90% aqueous solution).

10 g of the mixture obtained in this manner was applied to medium-blond natural hair. The hair was then covered with a plastic cap or plastic sheet. After a treatment time of 30 min at 40° C., 10 g of a 1:2 mixture of a persulfate-containing blonding powder and a 9% aqueous hydrogen peroxide solution was massaged in and allowed to act for 30 minutes. The hair was then rinsed with water, shampooed, rinsed with an acidic conditioner and finally dried.

The hair assumed a uniform bright gold-yellow color.

Unless otherwise indicated, the percentages given in the present patent application are by weight.

1-12. (canceled)

13. An agent for dyeing hair, said agent having a pH of 4 to 11 and containing at least one vat dye that is reduced by a reducing agent compound, said reducing agent compound forming an enediol in an alkaline medium.

14. The agent as defined in claim 13, wherein the reducing agent compound is selected from the group consisting of monohydroxyacetone, dihydroxyacetone, acetoin, glutaroin, adipoin, glycol aldehyde, benzoin, 2,3-dihydroxyacrylaldehyde, and cyclopentadiolone.

15. The agent as defined in claim 13, further comprising the reducing agent compound in an equimolar amount in relation to the at least one vat dye or in an up to 50-fold excess in relation to the at least one vat dye.

16. The agent as defined in claim 13, wherein said at least one vat dye is selected from the group consisting of Vat Yellow 1, C.I. Vat Yellow 2, C.I. Vat Yellow 3, C.I. Vat Yellow 4, C.I. Vat Yellow 12, C.I. Vat Yellow 13, C.I. Vat Yellow 17, C.I. Vat Yellow 20, C.I. Vat Yellow 26, C.I. Vat Yellow 28, C.I. Vat Yellow 33, C.I. Vat Yellow 46, C.I. Vat Orange 1, C.I. Vat Orange 2, C.I. Vat Orange 3, C.I. Vat Orange 7, C.I. Vat Orange 9, C.I. Vat Orange 11, C.I. Vat Orange 15, C.I. Vat Orange 17, C.I. Vat Orange 19, C.I. Vat Red 10, C.I. Vat Red 13, C.I. Vat Red 14, C.I. Vat Red 15, C.I. Vat Red 18, C.I. Vat Red 23, C.I. Vat Red 28, C.I. Vat Red 32, C.I. Vat Red 35, C.I. Vat Violet 1, C.I. Vat Violet 10, C.I. Vat Violet 15, C.I. Vat Violet 16, C.I. Vat Blue 4, C.I. Vat Blue 6, C.I. Vat Blue 20, C.I. Vat Blue 21, C.I. Vat Blue 25, C.I. Vat Blue 26, C.I. Vat 29, C.I. Vat Blue 30, C.I. Vat Blue 43, C.I. Vat Blue 64, C.I. Vat Blue 66, C.I. Vat Green 1, C.I. Vat Green 3, C.I. Vat Green 9, C.I. Vat Green 11, C.I. Vat Green 12, C.I. Vat Brown 1, C.I. Vat Brown 3, C.I. Vat Brown 45, C.I. Vat Black 16, C.I. Vat Black 25, C.I. Vat Black 27, C.I. Vat Black 29, and mixtures thereof.

17. The agent as defined in claim 13, containing from 0.01 to 10 weight percent of said at least one vat dye.

18. The agent as defined in claim 13, further comprising a cationic compound.

19. The agent as defined in claim 18, wherein said cationic compound is selected from the group consisting of Polyquaternium-2, Polyquaternium-4, Polyquaternium-5, Polyquaternium-6, Polyquaternium-7, Polyquaternium-10, Polyquaternium-11, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-31, Polyquaternium-35, Polyquaternium-36, Polyquaternium-37, Polyquaternium-39, Polyquaternium44, Polyquaternium46, Polyquaternium47, Polyquaternium-51, Polyquaternium-55, Polyquaternium-57, Quaternium-80, hydroxypropylguarhydroxypropyltrimethylammonium chloride, guar-hydroxypropyl-trimethylammonium chloride, and mixtures thereof.

20. The agent as defined in claim 18, containing from 0.001 to 5 weight percent of said cationic compound.

21. The agent as defined in claim 13, further comprising at least one developer compound and at least one coupler compound.

22. The agent as defined in claim 13, further comprising at least one self-coupling compound.

23. The agent as defined in claim 13, further comprising at least one natural or synthetic direct dye.

24. A method of dyeing hair, said method comprising the steps of:
a) pre-reducing at least one vat dye with a reducing agent compound to form at least one reduced vat dye, said reducing agent compound having an enediol form in a strongly alkaline medium having a pH of from 10 to 13;
b) providing an agent for dyeing the hair having a pH of from 4 to 11 and containing said at least one reduced vat dye; and
c) applying the agent for dyeing the hair to the hair.

25. The method as defined in claim 24, further comprising allowing the agent applied during the applying to act on the hair at 15 to 60° C. for a treatment time of 1 to 60 minutes and then re-oxidizing the at least one reduced vat dye with atmospheric oxygen or with an oxidant to form an insoluble pigment.

26. The method as defined in claim 25, wherein the oxidant is selected from the group consisting of hydrogen peroxide; addition compounds of hydrogen peroxide with urea, melamine, and sodium borate; persulfates; and mixtures thereof.

27. The method as defined in claim 25, further comprising protecting said at least one reduced vat dye from atmospheric oxygen during the allowing of the agent to act on the hair by covering the hair with a plastic cap.
